(19) United States
(12) Patent Application Publication
(10) Pub. No.: US 2024/0180660 A1
(43) Pub. Date: Jun. 6, 2024

(54) Drapes for Use in Mechanical Tissue Resuscitation Systems

(71) Applicant: Renovo Concepts, Inc., San Antonio, TX (US)

(72) Inventor: Larry Daniel DICK, San Antonio, TX (US)

(21) Appl. No.: 18/528,772

(22) Filed: Dec. 4, 2023

Related U.S. Application Data
(60) Provisional application No. 63/430,257, filed on Dec. 5, 2022.
(60) Provisional application No. 63/430,335, filed on Dec. 5, 2022.

(51) Int. Cl. A61B 46/00; A61B 46/20; A61F 13/05; A61F 13/12; A61M 1/00

(52) U.S. Cl. CPC A61B 46/40; A61F 13/05; A61F 13/12; A61B 2046/205; A61M 1/912; A61M 1/917

(57) ABSTRACT

A drape for covering and sealing a porous layer of material over a body of tissue being subjected to a decompressive procedure. The drape includes a sheet of silicone rubber material formed in a generally hemispherical shape with circular perimeter edge and a strip or band of adhesive material positioned on the circular perimeter edge of the hemispherical sheet. The drape may be positioned to cover the porous layer of material that is positioned on the body of tissue to seal the drape and the porous layer to the body of tissue. The drape is configured to be fitted with a port through which suction may be drawn.

### CROSS REFERENCES TO RELATED APPLICATIONS

This application claims the benefit under Title 35 United States Code § 119(e) of U.S. Provisional Patent Application Ser. No. 63/430,257; Filed: Dec. 5, 2022, and U.S. Provisional Patent Application Ser. No. 63/430,335; Filed: Dec. 5, 2022, the full disclosures of which are incorporated herein by reference.

### BACKGROUND OF THE INVENTION

### 1. Field of the Invention

The present invention has application in cases where a clinician is performing a decompressive procedure on a patient's skull and brain, where a mechanical tissue resuscitation manifold and evacuation dome are applied, and where the scalp cannot be sutured closed over the manifold and dome to create a seal. The hemispherical drapes of the present invention are applied in place of the sutured scalp, in order to form a seal over the area of treatment. The expandable drapes of the present invention are also applied in place of the sutured scalp, in order to effect a seal over the area of treatment. The expandable drapes effect a standardized one-size-fits-all cover seal, allow the seal to be effected faster, and allow the tension on the patient's head created by the drape to be adjusted for comfort.

### 2. Description of the Related Art

The present invention is intended to be used in some instances with the Mechanical Tissue Resuscitation Systems and Methods generally described in U.S. Pat. No. 8,267,960, Issued: Sep. 18, 2012, Title: Device and Method for Treating Central Nervous System Pathology; and in U.S. Pat. No. 8,764,794, Issued: Jul. 1, 2014, Title: Device and Method for Treating Central Nervous System Pathology; the full disclosures of which are each incorporated herein by reference.

In the past, scalp suturing or sheet drapes were cut to fit around the region, to create an ad-hoc seal.

### SUMMARY OF THE INVENTION

In cases where a clinician performs a decompressive procedure on a patient's skull and brain, where a mechanical tissue resuscitation manifold and evacuation dome are applied, and where the scalp cannot be closed over the manifold and dome to create a seal, the drape of the present invention is applied in place of the sutured scalp, in order to effect a seal over the area of treatment.

The hemispherical drape of the present invention effects a standardized cover seal, allows the seal to be effected faster, and allows the tension on the patient's head created by the drape to be mitigated based on the appropriate size of the drape chosen by the clinician. Use of the hemispherical drape will also effect more efficient subsequent patient brain access, as no sutures would need to be removed to access the site of therapy, if such access is required.

The expandable drape of the present invention effects a standardized one-size-fits-all cover seal, allows the seal to be effected faster, and allows the tension on the patient's head created by the drape to be adjusted for comfort. Use of the expandable drape will also effect more efficient subsequent patient brain access, as no sutures would need to be removed to access the site of therapy, if such access is required.

### BRIEF DESCRIPTION OF THE DRAWINGS

FIG. 1 is an exploded profile (side) partial cross-sectional view of an implementation of a first exemplary embodiment of the hemispherical drape of the present invention.

FIGS. 2A-2C are an isometric, partial cross-sectional view, a profile (side) partial cross-sectional view, and a detailed partial cross-sectional view of an implementation of the first exemplary embodiment of the hemispherical drape of the present invention.

FIG. 3 is an exploded, profile (side), partial cross-sectional view of an implementation of a second exemplary embodiment of the hemispherical drape of the present invention.

FIGS. 4A & 4B are a profile (side) partial cross-sectional view and a detailed partial cross-sectional view of an implementation of the second exemplary embodiment of the hemispherical drape of the present invention.

FIG. 5 is a perspective view of an implementation of a third preferred embodiment of the hemispherical drape of the present invention.

FIG. 6 is an exploded profile (side) view of an implementation of the third preferred embodiment of the hemispherical drape of the present invention.

FIG. 7A is a plan (top) view of a fourth exemplary embodiment of the present invention showing an expandable drape.

FIG. 7B is a plan (side) view of the fourth exemplary embodiment of the expandable drape of the present invention.

FIG. 7C is a perspective, profile (side) view of the fourth exemplary embodiment of the expandable drape of the present invention.

FIG. 7D is a perspective, assembly, profile (side) view of the fourth exemplary embodiment of the expandable drape of the present invention shown with a gusset in place.

FIG. 7E is a perspective, profile (side) view of the fourth exemplary embodiment of the expandable drape of the present invention.

FIGS. 8A-8D are an exploded perspective, an assembled perspective, and detailed profile (side) views, of an implementation of a fifth exemplary embodiment of the expandable drape and separate gusset of the present invention shown with the gusset removed and with the gusset in place.

FIGS. 9A-9C are an exploded perspective, an assembled perspective, and detailed profile (side) view, of an implementation of a sixth exemplary embodiment of the expandable drape and separate gusset of the present invention shown with the gusset removed and with the gusset in place.

FIGS. 10A-10D are an exploded perspective, an assembled perspective, and detailed profile (side) views, of an implementation of a seventh exemplary embodiment of the expandable drape and separate gusset of the present invention shown with the gusset removed and with the gusset in place.

### SUMMARY OF ELEMENTS OF THE INVENTION

### Summary of Structural & Compositional Elements (Hemispherical Drape)

(i) silicone (shore 40 A, 12 mil) molded into a hollow hemisphere.

(ii) silicone adhesive strip with peel-away paper cover.

### Summary of Structural & Compositional Elements (Expandable Drape)

(i) silicone (shore 40 A, 12 mil) molded into a hollow hemisphere.

(ii) silicone (shore 40 A, 8 mil) triangular or oblong insert (gusset).

(iii) silicone adhesive strip with peel-away paper cover.

### Summary of Methods of Use (All Embodiments)

(i) In mechanical tissue resuscitation therapy, applied to patient head after decompressive procedure and application of mechanical tissue resuscitation manifold.

(ii) In mechanical tissue resuscitation therapy, applied by clinician if the patient head cannot be sutured closed after application of mechanical tissue resuscitation.

(iii) Manifold and mechanical tissue resuscitation suction dome with attached tubing.

### Summary of Methods of Manufacture (Hemispherical Drape)

(i) Liquid silicone rubber (LSR) (shore 40 A) is heated to melting point in a bath.

(ii) A hemispherical dip mold (18 cm OD) is placed in the bath to form a layer of LSR onto the dip mold.

(iii) The dip mold is repeatedly placed into the bath in order to form a 12 mil thickness of LSR to the dip mold.

(iv) The molded hemispherical drape is removed from the dip mold.

(v) The silicone adhesive strip is applied to the inner circumferential edge of the drape.

### Summary of Methods of Manufacture (Expandable Drape)

(i) Liquid silicone rubber (LSR) (shore 40 A) is heated to melting point in a bath.

(ii) A hemispherical dip mold (18 cm OD) is placed in the bath to form a layer of LSR onto the dip mold.

(iii) The dip mold is repeatedly placed into the bath in order to form a L2 mil thickness of LSR to the dip mold.

(iv) ‘Ihe molded hemispherical drape is removed from the dip mold.

(v) A 10 cm slit is cut into the drape, normal to the leading edge of the drape.

(vi) The slit is separated to 10 cm at the edge of the drape.

(vii) A 10 cm wide by 10 cm long triangle is cut from a sheet of 8 mil silicone.

(viii) The cut triangle is fitted to the open slit of the drape.

(ix) The edges of the triangle are plastic welded (ultrasonic) to the edges of the slit to effect the insert.

(x) The silicone adhesive strip is applied to the inner circumferential edge of the drape and the insert.

### DESCRIPTION OF THE EXEMPLARY EMBODIMENTS

In cases where a patient undergoes a craniectomy or craniotomy, such that they are provided mechanical tissue resuscitation therapy, there is a need to seal the area where the disposable mechanical tissue resuscitation device is placed, in order to prevent ingress of contaminants and to provide a sterile means of maintaining an appropriate seal in which lossless suction can be applied through the mechanical tissue resuscitation disposable device. In some cases, suturing of the incised area, to close the skin around the mechanical tissue resuscitation device, may not be possible. The mechanical tissue resuscitation hemispherical drape of the present invention can be applied in such cases to create an overlying impermeable seal in order to maintain a sterile means of providing suction. The drape is shaped as a hemisphere, to provide a wrinkle-free overlay, and has an adhesive band applied to the inside diameter edge, which maintains the drape position on the head, and a lossless seal around the head. When applied over the mechanical tissue resuscitation manifold, an egress point can be made in the drape to allow passage of extracted fluids during suction. The adhesive mechanical tissue resuscitation suction dome is applied over the egress point, thus maintaining the sterile barrier established by the drape.

The expandable drape effects a standardized one-size-fits-all cover seal, allows the seal to be effected faster, and allows the tension on the patient's head created by the drape to be adjusted for comfort. Use of the expandable drape will also effect more efficient subsequent patient brain access, as no sutures would need to be removed to access the site of therapy, if such access is required. The size of the triangular insert may be adjusted during manufacture to effect greater or lesser expandability in the manufactured expandable drape. The insert could be removed entirely to eliminate the expandability option. The triangular insert could be inset into the cut flap to allow the addition of cinching tabs. The insert may be of different shapes, such as hemispherical, to allow the addition of large cinching tabs. Thicknesses of both the drape and insert could be adjusted during manufacturing to effect greater or lesser stiffness in the manufactured drape. The inner diameter edge adhesive strip could alternately be eliminated so that the drape edge would require external film tape to seal it to the scalp—external film tape is commonly used in the clinic for such purposes.

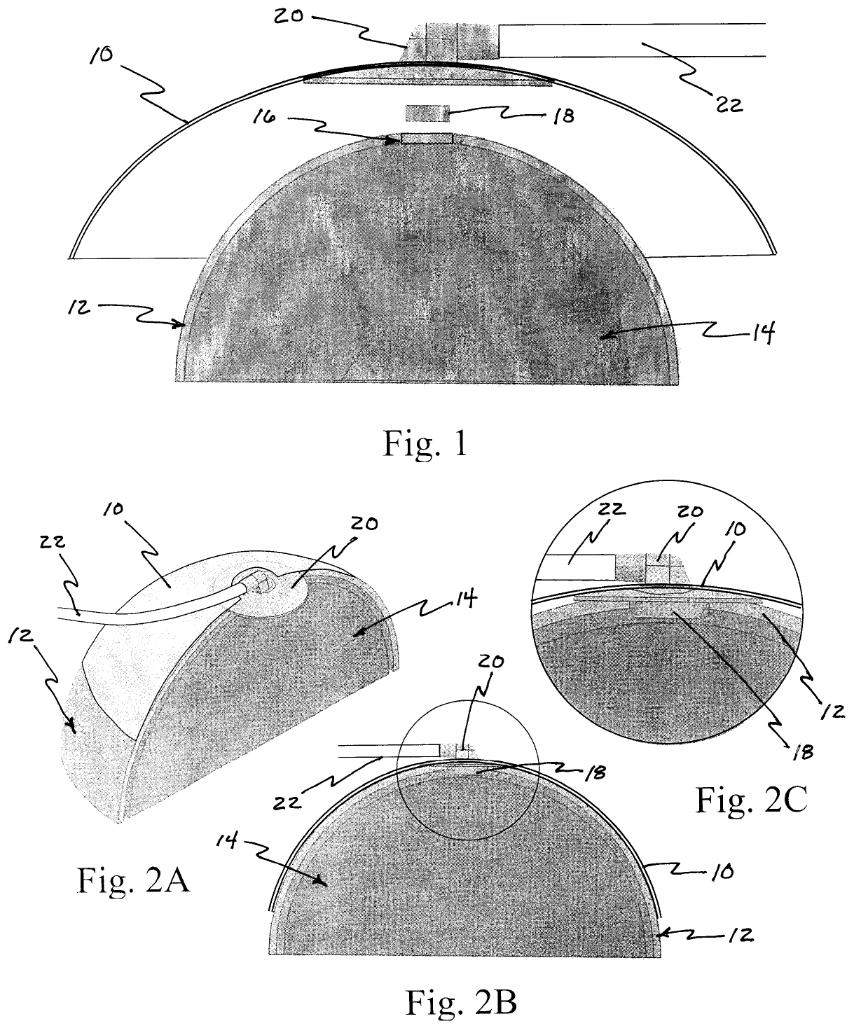

Reference is made first to FIG. 1 which is an exploded profile (side) partial cross-sectional view of an implementation of a first exemplary embodiment of the hemispherical drape of the present invention. In FIG. 1 hemispherical drape 10 is shown positioned on and sealed to disposable dome port 20 which is in turn connected to tubing 22 (⅛ inch ID typical). This assembly is then positioned over the patient's skull 12 and brain 14. In this first embodiment, a relatively small bore hole 16 is effected through the skull 12 to receive a sized disk of porous silicone foam 18 (17 mm diameter×6 mm thick typical).

FIGS. 2A-2C are an isometric partial cross-sectional view, a profile (side) partial cross-sectional view, and a detailed partial cross-sectional view of an implementation of the first exemplary embodiment of the hemispherical drape of the present invention. In FIG. 2A, hemispherical drape 10 is shown fixed on and sealed to dome port 20 (connected to tubing 22) and the assembly is then positioned on the patient's skull 12 as shown. FIG. 2B more clearly shows these same elements in the layered and sealed arrangement described. FIG. 2C provides details on the circled region in FIG. 2B. In FIG. 2C, the placement of silicone foam disk 18 between the brain tissue and suction at port 20 is shown. Drape 10 seals port 20 onto skull 12 and therefore directs suction only through the silicone foam disk. In most embodiments herein, drape 10 is preferably constructed of transparent material.

As suggested above, a 17 mm×6 mm silicone foam disk or puck works with a mechanical tissue resuscitation dome having a 48 mm diameter adhesive pad. Various other sizes work with a mechanical tissue resuscitation port that has a different sized adhesive pad. Alternately, the dome may be the same size for a variety of applications. It so happens that it covers the 17 mm puck completely, but would likely not cover a larger craniotomy application, potentially 8 inches in diameter, or larger. The drape in the second embodiment described below makes up the difference in the size of the dome pad, as it is also impermeable and adhesive. If the manufacture takes the bore-hole model and increases the size of the skull excision and manifold puck to an 8 inch diameter, the craniotomy model is effectively achieved.

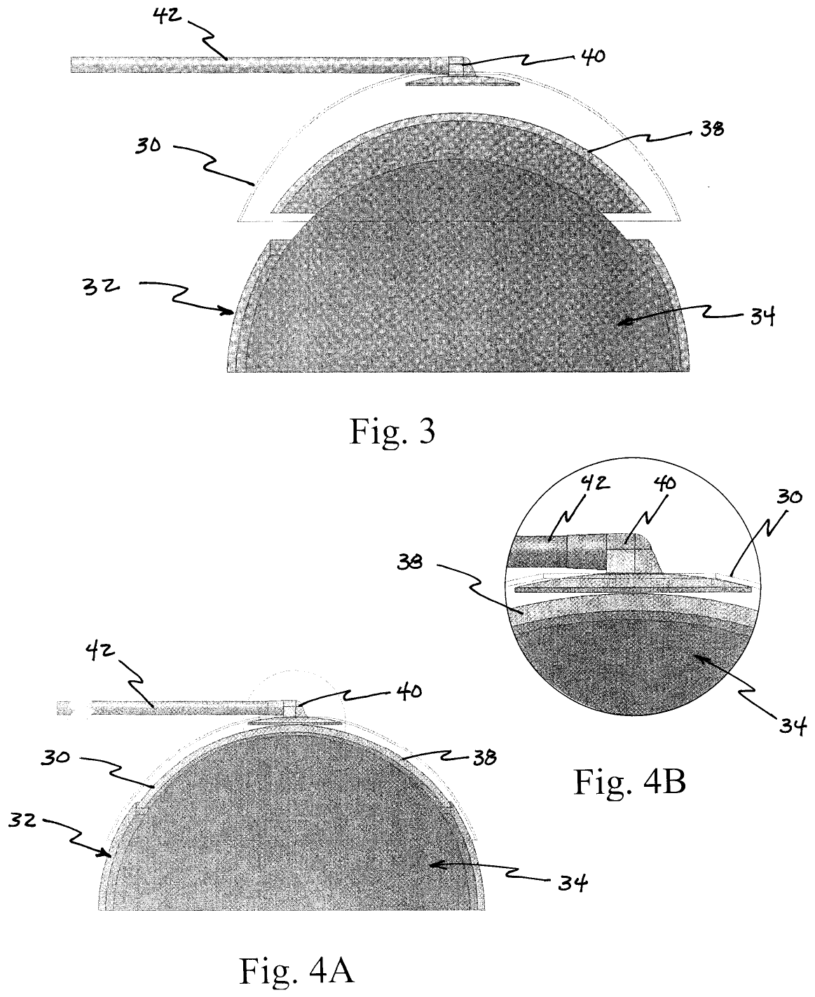

FIG. 3 is an exploded, profile (side), partial cross-sectional view of an implementation of a second exemplary embodiment of the hemispherical drape of the present invention. In FIG. 3 hemispherical drape 30 is shown positioned on and sealed to disposable dome port 40 which is in turn connected to tubing 42 (⅛ inch ID typical). This assembly is then positioned over the patient's skull 32 and brain 34 as in the first embodiment. In this second embodiment, a larger portion of the skull 32 has been removed as part of the specific mechanical tissue resuscitation procedure and a larger (8 inch×8 inch×3 mm typical) section of porous silicone foam 38 is positioned in contact with the tissue of the brain 34.

FIGS. 4A & 4B are a profile (side) partial cross-sectional view and a detailed partial cross-sectional view of an implementation of the second exemplary embodiment of the hemispherical drape of the present invention. In FIG. 4A, hemispherical drape 30 is shown fixed on and sealed to dome port 40 (connected to tubing 42) and the perimeter edge of the assembly is then positioned on the patient's skull 32 as shown. FIG. 4B provides details on the circled region in FIG. 4A. In FIG. 4B, the placement of silicone foam layer 38 between the brain tissue and suction at port 40 is shown. Drape 30 seals port 40 onto foam layer 38 and at the same time seals the remaining surface area of the foam layer 38 into contact with the tissue of the brain 34, sealing the same to the skull 32 at the perimeter of the drape 30. This assembly therefore directs suction only through the silicone foam layer 38 from the brain tissue 34. As in the previous embodiment, drape 30 is preferably constructed of transparent silicone material.

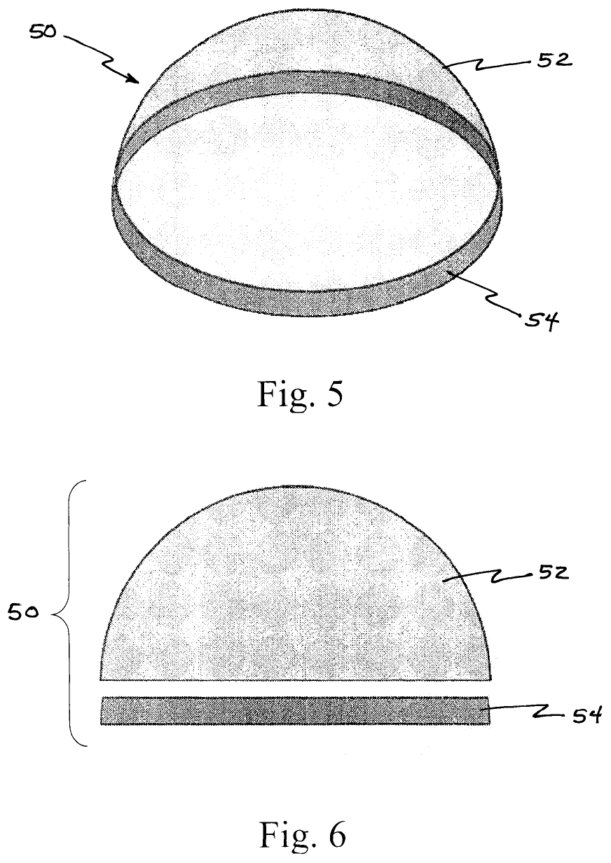

FIG. 5 is a perspective view of an implementation of a third preferred embodiment of the hemispherical drape 50 of the present invention showing an adhesive strip 54 positioned on or incorporated into the perimeter edge of the drape material 52. FIG. 6 is an exploded profile (side) view of this same implementation of the third preferred embodiment of the hemispherical drape of the present invention.

The size (diameter) of the hemispherical drape could be adjusted during manufacturing to effect greater or lesser choices in the size ranges of the manufactured drape. The size (thickness) of the drape could also be adjusted during manufacturing to effect greater or lesser stiffness (stretchability) in the manufactured drape. Nominal thickness would preferably be 0.5 mm. The inner diameter edge adhesive strip could be eliminated so that the drape edge would require external film tape to seal it to the scalp.

Drape 50 may preferably be manufactured in three or more preselected sizes; 16 cm, 18 cm, and 20 cm in diameter. An 18 cm diameter hemispherical drape equates with an approximate 57 cm circumference at the lower perimeter of the drape. The adhesive strip 54 positioned on or incorporated into the perimeter edge of the drape may preferably be about ½ inch wide.

Various alternative structures for the hemispherical drape of the present invention are disclosed wherein the effective perimeter diameter of the drape may be expanded or varied as required during use. As mentioned above, the expandable drape versions of the present invention effect a standardized one-size-fits-all cover seal, allow the seal to be effected faster, and allow the tension on the patient's head created by the drape to be adjusted for comfort.

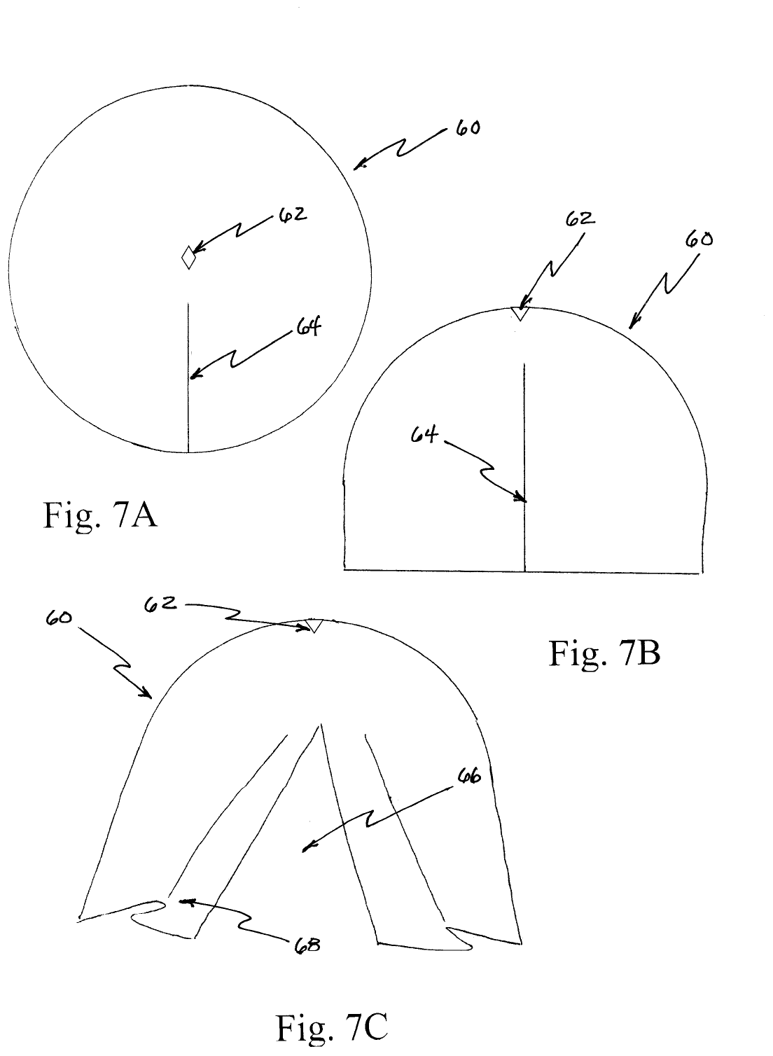

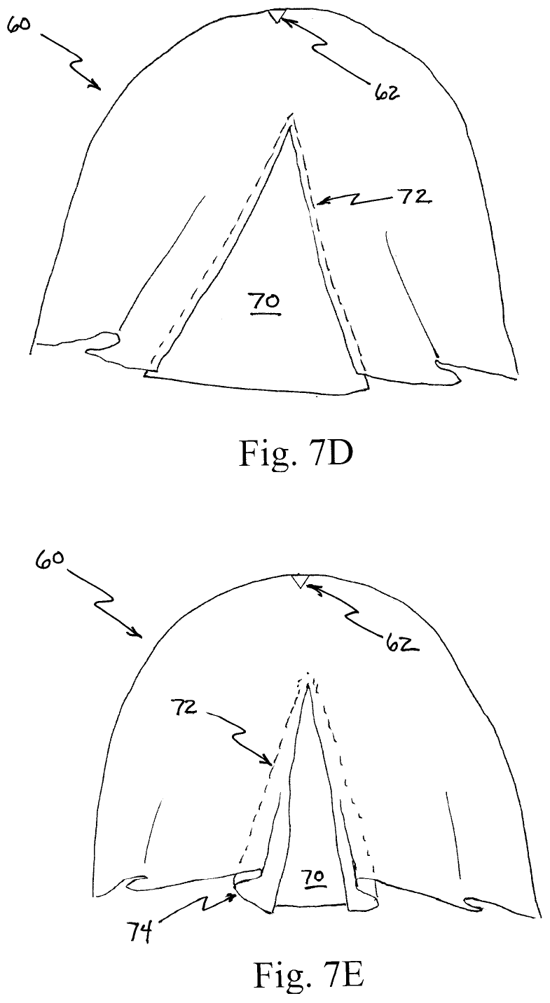

FIGS. 7A-7E show the basic structures and steps for producing an expandable drape of the present invention. FIG. 7A is a plan (top) view of a fourth exemplary embodiment of the present invention showing an expandable drape and FIG. 7B is a plan (side) view of the expandable drape. Drape 60 is hemispherical in form and incorporates an extraction port 62 and a cut slit 64. FIG. 7C is a perspective, profile (side) view of the fourth exemplary embodiment of the expandable drape of the present invention showing the step of opening the slit 64 forming slack folds 68 in preparation for positioning an expansion gusset.

FIGS. 7D & 7E continue the process steps from above with the placement of the expansion gusset. FIG. 7D is a perspective, assembly, profile (side) view of the fourth exemplary embodiment of the expandable drape 60 of the present invention shown with a gusset 70 in place. In this case the gusset 70 is preferably triangular in geometry although various other shapes may also be implemented. Triangular gusset 70 is preferably secured at ultrasonic (or other) weld seam 72 as shown. FIG. 7E is a perspective, profile (side) view of the fourth exemplary embodiment of the expandable drape 60 of the present invention showing the establishment of a flex-zone 74 on the gusset 70 that can be expanded or contracted as needed during use.

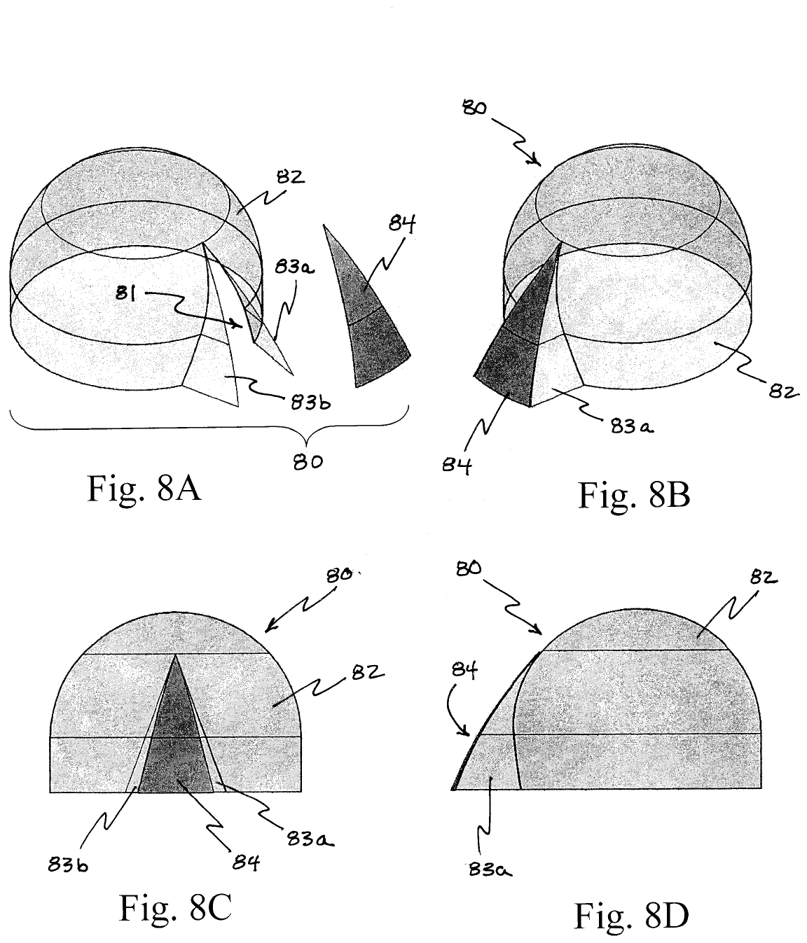

FIGS. 8A-8D are an exploded perspective, an assembled perspective, and detailed profile (side) views, of an implementation of a fifth exemplary embodiment of the expandable drape and separate gusset of the present invention shown with the gusset removed and with the gusset in place. Drape 80 is constructed of hemispherical drape 82 cut with a vertical slit to form opening 81 with flaps 83a & 83b. A separate triangular gusset 84 is formed and positioned onto the edges of flaps 83a & 83b. As seen in FIG. 8B, gusset 84 is welded to flap edges 83a & 83b to form expandable drape 80.

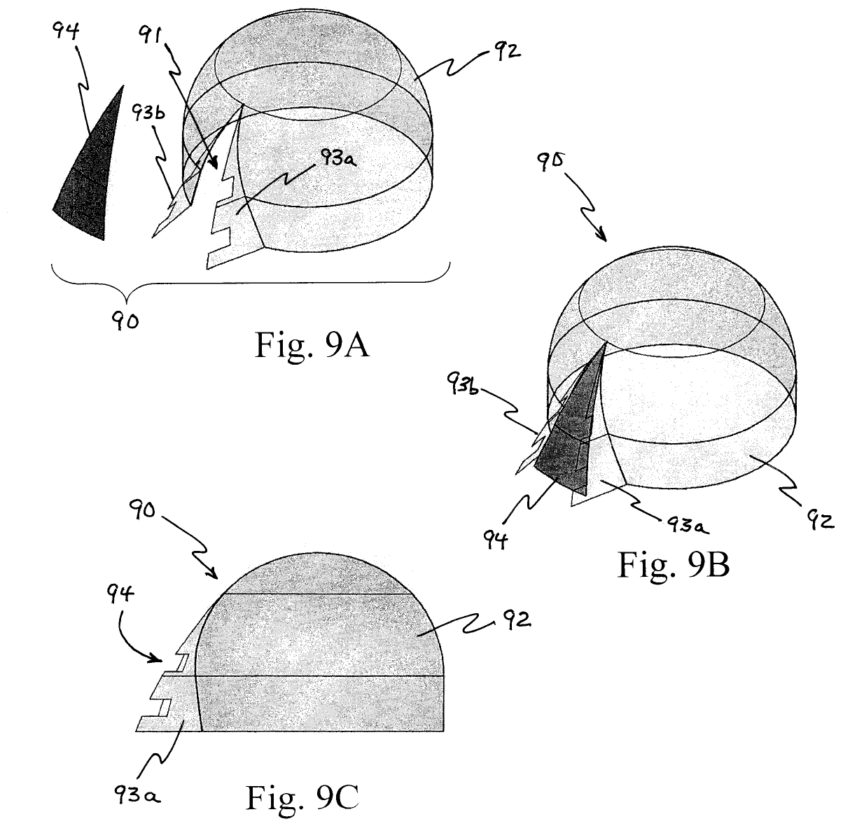

FIGS. 9A-9C are an exploded perspective, an assembled perspective, and detailed profile (side) view, of an implementation of a sixth exemplary embodiment of the expandable drape and separate gusset of the present invention shown with the gusset removed and with the gusset in place. Drape 90 is constructed of hemispherical drape 92 cut with a vertical slit to form opening 91 with flaps 93a & 93b. Tabs on 93a & 93b may be cut into the edges of the flaps or may be separate tabs added to the edges of the flaps. A separate triangular gusset 94 is formed and positioned onto the edges of flaps 93a & 93b. As seen in FIG. 9B, gusset 94 is welded to flap edges 93a & 93b on the inside edges of the tabs formed so that the tabs may be used to close or open the expandable drape 90 as needed. Preferably the tabs extend outward beyond the edges of flaps 93a & 93b to allow for the most expansion/contraction of the gusset filled gap in the perimeter edge of the drape.

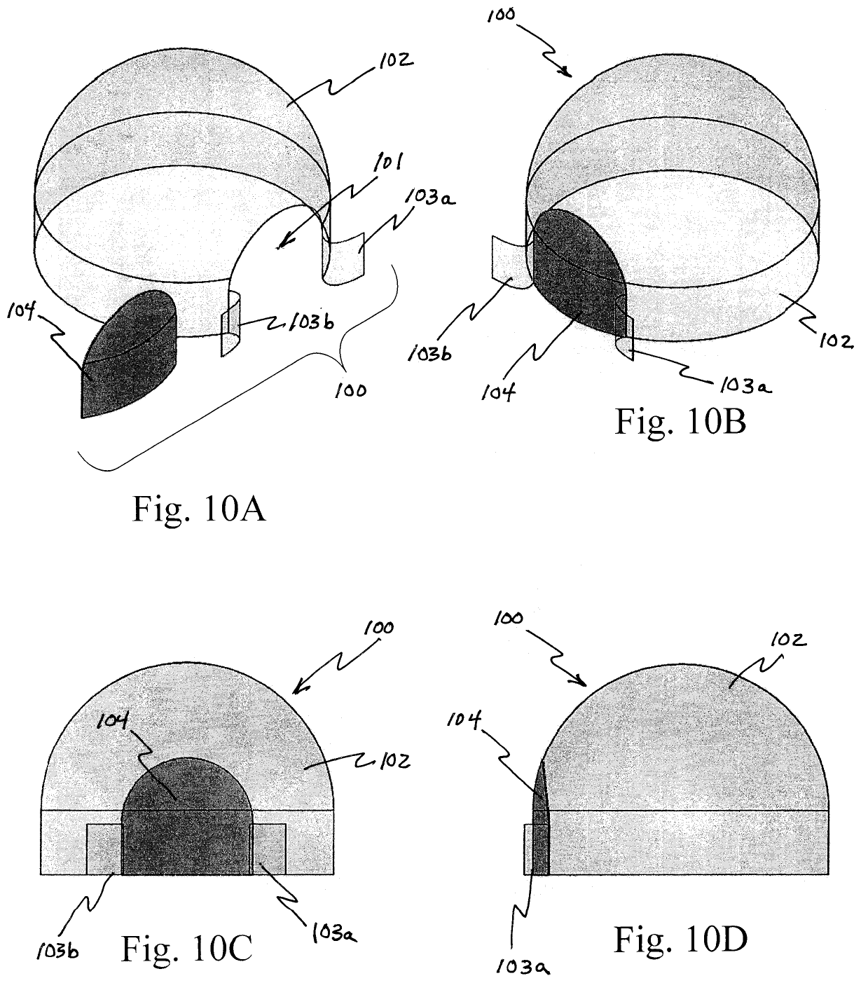

Finally, reference is made to FIGS. 10A-10D which are an exploded perspective, an assembled perspective, and detailed profile (side) views, of an implementation of a seventh exemplary embodiment of the expandable drape and separate gusset of the present invention shown with the gusset removed and with the gusset in place. Drape 100 is constructed of hemispherical drape 102 cut with a T-shaped opening slit (from the lower perimeter edge) and a semi-circular cutout (above the T-shaped opening slit) to form opening 101 with flaps 103a & 103b. Flaps 103a & 103b form useful tabs to expand or contract opening 101. A separate die cut fill panel 104 is formed and positioned onto the edges of flaps 103a & 103b as well as the inside edge of the semi-circular cutout. As seen in FIG. 9B, gusset 104 is welded to flap edges 103a & 103b on the inside edges of the tabs formed so that the tabs may be used to close or open the expandable drape 100 as needed. In this embodiment, the shape of the gusset 104 and the placement of the tabs facilitates a cinch-cap style of expandable drape. The basic design of this embodiment will also function with a single tab. The tabs in this case may preferably have adhesive strips on the inside surfaces as the rest of the inside diameter of the perimeter edge of the drape. As may be anticipated from the structures shown, this embodiment is produced with a maximum diameter geometry and may be cinched down to smaller diameters as needed. t expansion/contraction of the gusset filled gap in the perimeter edge of the drape.

### Summary of Features & Advantages

Applications include cases where the clinician performs a decompressive procedure on the patient skull and brain, where a mechanical tissue resuscitation manifold and evacuation dome are applied, and where the scalp cannot be sutured closed over the manifold and dome to create a seal, the hemispherical drape of the present invention can be applied in place of the sutured scalp, in order to effect a seal over the area of treatment. The drape effects a standardized cover seal, allows the seal to be effected faster and effects more efficient subsequent patient brain access, as no sutures would need to be removed to access the site of therapy, if such access is required.

### Alternate Embodiments of the Present Invention

The size (diameter) of the hemispherical drape could be adjusted during manufacturing to effect greater or lesser choices in the size ranges of the manufactured drape. The size (thickness) of the drape could be adjusted during manufacturing to effect greater or lesser stiffness (stretchability) in the manufactured drape. The inner diameter edge adhesive strip could be eliminated so that the drape edge would require external film tape to seal it to the scalp—external film tape is commonly used in the clinic for such purposes.

The size of the triangular gusset may be adjusted during manufacture to effect greater or lesser expandability in the manufactured drape. The insert could be removed entirely to eliminate the expandability option. The triangular insert could be inset into the cut flap to allow the addition of cinching tabs. The insert may be of different shapes, such as hemispherical, to allow the addition of large cinching tabs. Thicknesses of both the drape and insert could be adjusted during manufacturing to effect greater or lesser stiffness in the manufactured drape.

The size and shape of the slits and cutouts could be adjusted during manufacturing and/or during use. Slits alone or slits combined with cutouts may be utilized. Slits may be made to form flaps of varying sizes, again with or without the use of cutouts (see Drawing Figures) resulting in variations in expandability.

Although the present invention has been described in conjunction with a number of preferred embodiments, those skilled in the art will recognize modifications to these embodiments that still fall within the spirit and scope of the invention. The invention has been described in terms of a basic embodiment with a number of add-on functionalities that may be implemented separately or collectively. Further add-on functionalities will be anticipated by those skilled in the art that do not depart from the spirit and scope of the invention as set forth in the appended claims.

I claim:

1. A drape for covering and at least partially sealing a porous layer of material onto and over a surface of a body of tissue being subjected to a decompressive procedure, the drape comprising:
a sheet of silicone rubber material formed in a generally hemispherical shape having an inside surface and an outside surface, the hemispherical shaped silicone sheet having a generally circular perimeter edge, the hemispherical shaped silicone sheet sized incrementally larger than the porous layer of material; and
a strip of adhesive material positioned on the circular perimeter edge of the hemispherical shaped silicone sheet;
wherein the drape is configured to be positioned to cover the porous layer of material positioned onto and over the surface of the body of tissue to seal the drape and the porous layer to the body of tissue, wherein the drape is further configured to be fitted with a port through which suction may be drawn.
